(19) United States
(12) Patent Application Publication
(10) Pub. No.: US 2014/0128823 A1
(43) Pub. Date: May 8, 2014

(54) SYSTEM AND METHOD FOR TREATING COMPARTMENT SYNDROME

(71) Applicants: Rick M. Odland, Roseville, MN (US); Scott R. Wilson, Maple Grove, MN (US); Bradford G. Staehle, Minnetonka, MN (US)

(72) Inventors: Rick M. Odland, Roseville, MN (US); Scott R. Wilson, Maple Grove, MN (US); Bradford G. Staehle, Minnetonka, MN (US)

(73) Assignee: TWIN STAR MEDICAL, INC., Minneapolis, MN (US)

(21) Appl. No.: 14/127,549

(22) PCT Filed: Jun. 20, 2012
(86) PCT No.: PCT/US12/43353

§ 371 Date: Dec. 19, 2013

Related U.S. Application Data
(60) Provisional application No. 61/500,005, filed on Jun. 22, 2011.

(51) Int. Cl. A61M 1/00

(52) U.S. Cl. CPC A61M 1/0039
USPC 604/319

(57) ABSTRACT

A system for use in treating a tissue site, e.g., a site exhibiting or at risk for developing compartment syndrome, the system including a monitor for use in controlling the function and operation of one or more apparatuses providing insertable catheters. The system includes at least one apparatus having a catheter portion that includes both a suitably protected, functional device tip, adapted to be positioned and used within the tissue site, and one or more hollow fiber membranes, e.g., an array of such membranes, adapted to be positioned within the tissue site in order to simultaneously collect fluid therefrom. The catheter portion is adapted to be safely inserted and positioned within the tissue site, in a manner that permits it to function there while substantially minimizing direct impingement of the non-fluid tissue on the functional device tip surface.

### TECHNICAL FIELD

The present invention relates to the use of catheters for assaying, removing and/or providing fluids within or to the body. In another aspect, the invention relates to methods and apparatuses for monitoring parameters such as tissue pressure within the body by means of functional tips positioned in the tissue itself.

### BACKGROUND OF THE INVENTION

Various means exist for providing functional tips, e.g., sensors, within tissue, in order to determine corresponding tissue or body parameters such as tissue pressure, blood pressure, temperature, oxygenation, and the like.

Compartment syndrome is a condition that can occur following injury or in association with certain pathologic conditions, in which the pressure within a muscle compartment becomes elevated, due to tissue swelling, such that capillary blood flow is reduced below a level necessary to sustain tissue viability.

Normally, tissue swelling is constrained by an inelastic muscle covering called fascia. The excess fluid causes hydrostatic pressure within the injured compartment to increase significantly. As compartment pressure increases, the microvasculature can become compressed to the point where it eventually collapses, effectively reducing or stopping perfusion within the injured tissue and leading to ischemia of that tissue. The most common causes of compartment syndrome are trauma, including fractures, burns, or crush injuries. Compartment syndrome can also occur after vascular injury, reperfusion after ischemia, extravasation of fluid, and external compression.

Since compartment syndrome is often difficult to diagnose, and since the consequences of a misdiagnosis can be potentially severe, current clinical practice assumes that any patient who suffers a fracture of the lower leg is at risk for developing compartment syndrome. The difficulty in diagnosing compartment syndrome is due, at least in part, to the inability to distinguish between its clinical symptoms and other symptoms that are commonly seen after any tibial fracture. These difficulties can be exacerbated when the condition occurs under less than ideal conditions, such as in the course of combat.

Applicant has previously provided apparatuses and methods useful for assessing and treating compartment syndrome. See, for example, Applicant's pending US application having Serial No. U.S. Ser. No. 12/524,445.

### BRIEF DESCRIPTION OF THE DRAWING

FIG. 1 provides a side view of a catheter assembly including a pressure sensor and fluid collection catheter according to an embodiment of the invention.

FIG. 2 provides a detailed view of a distal section of the assembly of FIG. 1, configured for insertion.

FIG. 3 provides a detailed view of the distal end of the assembly of FIG. 1.

FIG. 4 provides a cross sectional view taken along line A-A of the assembly of FIGS. 1 and 2.

FIG. 5 provides a view of an alternative catheter assembly including both an oxygen sensor and fluid collection catheter according to another embodiment of the invention.

FIG. 6 provides a detailed view of the distal section of the assembly of FIG. 5, configured for insertion.

FIG. 7 provides a detailed view of a portion of the catheter assembly of FIG. 5, illustrating an oxygen sensor.

FIG. 8 provides a cross sectional view taken along line B-B of the assembly of FIGS. 5-7.

FIG. 9 provides a view of another catheter assembly including a removable cartridge according to an embodiment of the invention.

FIG. 10A provides a detailed view of the removable cartridge of FIG. 9.

FIG. 10B provides a cross sectional view taken along line C-C of the removable cartridge of FIG. 10A.

FIG. 11 provides a detailed view of a cartridge manifold section of the assembly of FIG. 9.

FIG. 12 provides a side view of a trocar for use in an embodiment of the invention.

FIG. 13 provides a side view of a fluid collection catheter for use in a system of this invention.

FIG. 14 provides a detailed view of the distal section of the catheter of FIG. 13, configured for insertion.

FIG. 15 provides a cross sectional view taken along D-D of the assembly of FIGS. 13-14.

FIG. 16 provides a detailed view of the distal end of the catheter assembly of FIGS. 13-14.

FIGS. 17A-17E provide views of a catheter assembly monitor according to an embodiment of the invention.

FIG. 18 provides a view of another catheter assembly monitor according to an embodiment of the invention.

FIG. 19 provides a view of the catheter assembly monitor of FIG. 18 in use in combination with multiple catheters of an assembly of an embodiment of the invention.

FIGS. 20A-20C provide front views of multiple catheter placements for various tibia fracture settings.

### SUMMARY OF THE INVENTION

In one embodiment, the present invention provides a system that includes one or more apparatuses comprising a catheter portion that, in turn, comprises both a functional device tip and one or more hollow fiber membranes (e.g., a “pressure monitor/fluid collection” catheter). The catheter permits the functional device tip, e.g., a sensor surface, to be positioned and used within a body, and in functional contact with a tissue site that comprises both fluid and non-fluid tissue. In preferred embodiment, the apparatus is adapted to be safely inserted and positioned within the tissue site, in a manner that permits it to function there (e.g., monitor pressure while also collecting fluid), while substantially minimizing direct impingement of the non-fluid tissue on the surface of the functional device tip.

In a particularly preferred embodiment, the pressure monitor/fluid collection catheter comprises a distal insertable portion that comprises a generally central pressure monitor (e.g., fiber optic cable and sensor), surrounded by a protective catheter, and in turn, by a plurality of hollow fiber membranes. More preferably, the apparatus further comprises one or more corresponding conduits and respective connection and/or communication points for use in delivering the pressure signal to the monitor, and for use in monitoring and controlling the withdrawal of fluid from the site. Fluid can be withdrawn in any suitable manner, and is preferably withdrawn by the application of negative pressure (e.g., vacuum) to the hollow fibers, e.g., in the form of either continuous or intermittent vacuum, and delivering the fluid to one or more corresponding chambers, in order to achieve a preventive or therapeutic effect, and optionally also for analytical purposes.

In a further preferred embodiment, the system comprises one or more additional apparatuses comprising fluid collection catheters, for use in combination with the pressure monitor/fluid collection catheter. Preferably, the fluid collection catheter(s) are adapted to be used in combination with the one or more pressure monitor/fluid collection catheters, e.g., being positioned in corresponding locations associated with the tissue site, the catheters being operated together in a manner that optimizes prevention and/or treatment. Either or both the PMFC and FC catheter(s) can be adapted to run on the same vacuum source, optionally in a manner that permits them to be separately controlled. One or more of the fluid collection catheter(s), in turn, can optionally and preferably provide additional functions or features, e.g., an insertable sensor for determining other body parameters, such as tissue oxygen levels or temperature.

In a particularly preferred embodiment, a monitor for use in a system of this invention provides one or more features designed to improve the function of this system as compared to those previously known. Preferably, these features are selected from the group consisting of: a) a self zeroing pressure transducer, b) one or more pressure alarms, c) wireless (e.g., WiFi) communication capability, d) data transmission capability, e) renewable and/or replaceable power sources (e.g., battery), f) all in one utility and mounting, and g) accessory USB inputs.

In medical devices that measure pressure, the pressure sensors or transducers typically measure absolute pressure, and in turn, generally need be initially “zeroed” with respect to atmospheric pressure. To accomplish this, the sensor/transducer typically is connected to a monitor prior to inserting into the patient, and atmospheric pressure is then measured and used as a reference (i.e., with atmospheric pressure establishing the baseline, or “zero” level). Applicant has discovered that the need to first zero the sensor can provide a disadvantage for systems such as the one presently claimed, in that the monitor will typically need to be brought into the operating room so that sterile catheters can be connected to the monitor prior to inserting into the patient. In turn, Applicant has discovered the manner in which a monitor for use in a system of this invention can instead be “self zeroing”, e.g., by incorporating a barometer inside the monitor. The monitor is designed to measure the atmospheric pressure from the barometer, in order to then subtract that measurement from the catheter pressure sensor. This eliminates the need to initally “zero” the sensor according to ambient conditions, as it is automatically performed by the monitor. At least one key advantage includes the fact that the catheters can now be placed in surgery, and the monitor can be connected at any time. Another key advantage is that the monitor can now continuously “calibrate” according to changes in ambient pressure. Specifically, when monitoring patients that are exposed to significant changes in ambient pressure (i.e. during aeromedical transport), the monitor can adjust the “Zero” reference according to the current ambient pressure.

In addition, a monitor of this invention will preferably include one or more pressure alarms, e.g., to permit the clinician to set notification limits for intra muscular pressure (IMP), and perfusion pressure (PP). Once limits have been entered, the monitor will display visible and/or audible alarms when limits are reached. In a preferred embodiment, a system also permits patient data to be transferred to one or more external devices for real time and/or future analysis, e.g., by use of a USB flashdrive, LAN cable, or wireless data transmission (e.g., through a WiFi network). A preferred monitor will also provide suitable communication means (e.g., WiFi antenna), together with means for sending data to an IP (internet protocol) address, in order to permit data to be accessed by clinicians within the corresponding network. Users will be able to see the same pressure display from a remote location within the wireless network. Additionally, a preferred monitor will indicate when it is in a “Battery Powered” mode, and have a battery power indicator, display approximate battery life. When battery life is low, the battery pack is preferably capable of being replaced and/or recharged.

Similarly, a preferred monitor of this invention will provide an “all in one” mounting system, permitting it to be mounted to IV pole, bed rail, and air transport mounting systems. The monitor will be capable of connecting accessory devices via USB port (e.g., near infrared spectroscopy “NIRS” devices). Accessory devices will be displayed through the monitor, and data will be stored.

The present application describes the manner in which an apparatus as described in Applicant's U.S. Ser. No. 12/524,445, the contents of which are incorporated herein by reference, while suitable for use in many indications, can nevertheless be modified and improved upon in order to provide one or more additional or different features or functions, which in turn, can further and expand the usefulness of an apparatus and/or corresponding system, including for use under demanding conditions such as use in the field of military combat.

In one preferred embodiment, the invention provides a system that comprises one or more pressure monitor/fluid collection catheters, and optionally, one or more fluid collection catheters, together with means for providing a vacuum source, and means for both monitoring, and in turn, controlling the function of the system. In turn, the various catheters can be both individually and collectively positioned and controlled in a manner that optimizes patient treatment. In a particularly preferred embodiment, the PMFC and FC catheters are adapted to be connected to each other, and in turn, to a single vacuum source, in order to simultaneously and/or individually controlled to withdraw fluid, and provide a preventive or therapeutic effect.

### DETAILED DESCRIPTION

An apparatus of the present invention can change the standard of care for military orthopedic injuries, since it can provide potential preventive and therapeutic benefits of a compartment monitoring device in the treatment of soldiers having extremity injuries and either exhibiting or at risk for developing compartment syndrome.

The terms “fluid” and “non-fluid” tissues, as used in this context, will generally refer, respectively, to the difference between tissue that is intended and able to be take up by, or into, an apparatus of this invention, in order to be measured, removed, etc., at or by the functional device tip, as compared to tissue(s) that instead have the tendency or risk of impinging upon or occluding such an apparatus, to the point where a functional device tip can not be used for its intended purpose.

In a preferred embodiment, the apparatus comprises one or more sensors within a catheter portion of the apparatus, for use in determining one or more corresponding parameters such as tissue pressure, the catheter being configured and used in a manner that permits the catheter portion to be positioned in tissue in a manner that permits the sensor(s) to effectively contact fluid tissue within the site, but that minimizes the extent to which the sensor(s) can be impinged upon by non-fluid tissue itself.

In a particularly preferred embodiment, the apparatus also includes one or more conduits, e.g., solid conduits or semipermeable membranes, which provide the ability to either deliver and/or remove fluids and/or components thereof, to or from the tissue site, as for therapeutic and/or analytical purposes. In one such preferred embodiment, the invention provides one or more sensors (e.g., a pressure sensor) adapted to be positioned and used within the tissue site itself, without substantial impingement or occlusion by non-fluid tissue that may be present. Preferably, the catheter also provides the ability to deliver materials (e.g., active agents) and/or remove fluid from a point within the tissue site (e.g., proximal or distal to the sensor surface), thereby providing improved clinical relevance as compared to conventional apparatuses in which both functions, and corresponding structures, are not integrated in a single apparatus.

Alternatively, the apparatus itself is considered novel, and can be used, solely for the purpose of improved sensor placement and use. More preferably, the catheter of this invention provides both improved sensor placement, protection, and use, as well as fluid delivery/removal means, and in turn, corresponding options adapted to improve its utility, and in turn, clinical relevance.

An apparatus of this invention permits the use of a sensor needing to have fluid or other communication with a body site, to be placed within or amidst tissue itself, in a manner that substantially prevents non-fluid tissue from impinging upon the sensor surface, while ensuring patency and optimal performance in the course of its placement and use.

In one preferred embodiment, the sensor tip is placed sufficiently back (proximal) from an exposed tip of the apparatus, in order to let fluid access the sensor itself, while substantially preventing contact with or impingement upon non-fluid tissue. In turn, in order to avoid the creation of an air bubble or other artifact within the exposed apparatus tip, one or more suitable vents are preferably positioned between the sensor itself and the apparatus tip, thereby permitting what little air or artifact there may be in the apparatus tip to escape as the tip itself is filled with fluid.

The apparatus comprises one or more sensors that can be positioned within the catheter, and in turn, within the tissue site, in a manner that permits its use without occluding necessary openings or pores, or direct tissue impingement. In one preferred embodiment, the catheter permits a pressure or other suitable sensor to itself remain separated from direct tissue contact, yet in sufficient fluid communication with the relevant tissue site, in order to permit pressure to be measured accurately, yet not in direct contact with the tissue site itself. For instance, in one preferred embodiment the pressure sensor is a fiber optic sensor incorporating white-light polarization interferometry technology, and is encased within the apparatus tip within a protective material adapted to protect the sensor tip, yet permit pressure to be accurately transferred to it from surrounding fluid.

As seen in the Drawing, for instance, the sensor, including the surface thereof, can be encased in a suitable substance (e.g., silicone gel). The substance provides sufficient physical parameters (e.g., stability, stiffness) to permit pressure to be accurately transferred to, and hence sensed by, the sensor surface. Sensors for use in a catheter of this invention can be of any suitable type and configuration, e.g., for use in monitoring pressure, pH, temperature, oxygenation, potassium or other electrolytes, biomarkers, optical spectroscopy parameters, tissue impedance, and so on. Optionally, or additionally, the sensor can have or provide a functional aspect as well, e.g., by providing heat, ultrasound and/or an electrical signal sufficient to treat the corresponding tissue site or surface (e.g., by breaking up clots, electroporation, and the like).

Suitable sensors can be based on any technology, e.g., fiber optic, electronic chips, ultrasound, and are preferably fiber optic based sensors adapted to by means of ‘white light interferometry’. See, for instance, “Miniature Fiber Optic Pressure Sensor for Medical Applications: an Opportunity for Intra-Aortic Balloon Pumping (IABP) Therapy”, E. Pinet et al. and “Opsens White-light Polarization Interferometry Technology”, Opsens, Inc., the entire disclosures of both of which are incorporated herein by reference.

Suitable fiber optic sensors are commercially available, e.g., as the “FOP-MIV” sensor available from Fiso Technologies, Inc., which is described as a front looking sensor that allows in situ measurements at locations unreachable to standard pressure sensors. Suitable fiber optic sensors provide an optimal combination of such features as durability and reliability, low cost, ease of use, miniature size, mounting flexibility, resolution, consistency, accuracy and precision, reading rate, fast response, low drift value, and the ability to provide a clear definition of complex pressure waveforms, as well as immunity to electromagnetic field or radiofrequency interference.

The apparatus preferably further comprises one or more lumen for delivering and/or removing fluid from the tissue site, preferably by means of one or more hollow fibers, and more preferably further comprises one or more vent or shunting means located distally, in order to permit sampling and/or removal of fluids having optimal clinical relevance.

In a particularly preferred embodiment, the apparatus includes a functional tip provided by a catheter that further comprises one or more conduits which provide the ability to either deliver and/or remove fluids and/or components thereof, for therapeutic and/or analytical purposes. In turn, it is quite preferable that the timing and position of the delivery and/or removal of fluids (including components) to or from the tissue site is integrated with the location and function of the functional tip, e.g., such that fluids or active agents intended to alleviate tissue swelling are removed from and/or delivered to the tissue site, in a manner that corresponds with readings generated by the pressure sensor itself.

A preferred apparatus of the present invention can include, for instance, the use of a suitable sensor associated with, while also positioned and protected at, the distal end of an optical fiber and within a distal portion having slits or other suitable means for permitting fluid communication between the sensor and tissue surrounding the distal portion. In one embodiment, the sensor can be displaced back from the most proximal portion of the slits. The tubular distal portion serves to protect the sensor from direct tissue impingement. The slit prevent occlusion of the tube and maintains communication contact with the tissue pressure, due to the bending of the slits when impinging tissue, allowing the sensor fluid contact through the slits.

In an alternative preferred embodiment, the distal portion of the apparatus is provided with a plurality of apertures for permitting fluid communication between the sensor and tissue surrounding the distal portion. As with the use of slits, the apertures prevent occlusion of the tube and maintain communication contact with the tissue pressure, allowing air to vent from the tip, and in turn, permitting the sensor fluid contact throughout the apparatus tip.

An apparatus of this invention can be prepared using any suitable techniques, e.g., the various parts can be provided separately and assembled in a suitable manner. Alternatively, various combinations and subcombinations of parts can be provided as integral parts, to be finally assembled with others.

The sensors, e.g., fiber optic sensors, and other components for use in the apparatus of this invention can include miniature, micro- and even nanotechnology components for use in minimally invasive diagnosis, therapy, and monitoring, including for instance, physical sensors that are linked to a telemetric unit for wireless data transmission. Such sensors can be biocompatibility packaged or implanted and used in a minimally invasive procedure, to determine such parameters as pressure and/or constituent levels in the blood or tissue itself, temperature, and/or tissue (e.g., nerve) function, and other suitable biological parameters.

An apparatus of this invention can be used, for instance, for the removal of interstitial fluid in order to lower muscle compartment pressure and thereby possibly reducing the need for surgical fasciotomy. In turn, the apparatus can be used in any suitable tissue site, and typically muscle site, including muscles of the arm or leg (e.g., anterior, posterior, deep posterior and/or lateral compartments). For instance, patients that have suffered an isolated tibial fracture (open or closed) typically require surgical stabilization within 72 hours of injury. Such patients can be treated with one or more apparatuses of this invention, which can be inserted at the end of the surgical procedure to stabilize the tibia fracture and can be connected to the pressure monitor before leaving the surgical room.

The apparatus(es) can be used for the first hours or days following surgical fixation of the affected lower leg in order to: (1) measure and record muscle compartment pressure, and (2) remove accumulated interstitial fluid. Such patients can be treated in any suitable manner, for instance, receiving constant or intermittent vacuum, at the same or varying levels, and optionally, in combination with fluid removal. The apparatus can be provided as either a single-fiber catheters or as multi-fiber catheters.

Situations in which the fluid removal capabilities are employed will typically result in a greater reduction in muscle compartment pressure, as compared to monitoring alone. Samples of the interstitial fluid removed from the patient's leg can be analyzed for various indicators of muscle injury, as well as to determine the serum levels of the same targeted analytes. Interstitial fluid and blood serum levels of the analytes can be correlated to intramuscular pressure levels and other parameters as well.

In such an embodiment, a system of this invention can include at least four components, including, an introducer, an apparatus for pressure monitoring and fluid collection, one or more fluid collection catheters, and a suitable compartment pressure monitor.

A preferred monitoring/collection apparatus for use in the present invention can monitor muscle compartment pressure as well as facilitate excess fluid removal, and is provided as a sterile disposable. In a particularly preferred embodiment, the apparatus includes a catheter body, hollow fiber membrane, a fiber optic pressure sensor, vacuum line and pressure sensor connectors, a catheter connection manifold, and a fluid sampling chamber with collection port. The apparatus can be used to provide various functions, including to measure compartment pressure, to remove interstitial fluid, to provide fluid samples for analysis, and to provide connections for additional fluid collection catheters.

In one embodiment, a preferred apparatus contains a pressure sensor at the distal tip that measures compartment pressure throughout the treatment period. The sensor can be connected to the monitor module by any suitable means, e.g., by wireless connection and signal or by an optical fiber that extends through the entire length of the catheter. A fiberoptic pressure connector is shown located at the proximal end of the apparatus, for use in connecting to a monitor.

A manifold can be located just proximal to the fluid collection chamber. Two additional fluid collection (FC) catheters can be connected to the manifold using standard luer connections. An FC catheter can be designed and used to provide additional fluid collection locations within the same compartment as a monitoring/collection apparatus. The FC catheters, in turn, will typically not provide pressure measurement and are designed and intended for use with a monitoring or monitoring/recovery apparatus. The FC catheter can include a catheter body, hollow fiber membrane, vacuum line connector, and a fluid sampling chamber with collection port. Interstitial fluid is removed through the hollow fiber membrane located at the distal section of the apparatus. Fluid passes through the walls of the micro-porous membrane, through the apparatus body and into the fluid collection chamber. The fluid collection chamber is connected to a vacuum line, which connects to the manifold of the apparatus. The monitor can be used to provide an intermittent, variable, and/or constant vacuum to the apparatus to draw fluid through the hollow membrane. Fluid that is contained in the collection chamber can be aspirated using a standard syringe through the collection port. The fluid can be transferred to a vial and saved for analysis.

The apparatus is designed to be used with the monitor, which can sense, display and record compartment pressure as measured by one or more such apparatuses. In addition, the monitor can be used to measure patient blood pressure using the cuff provided, which is used for calculating the perfusion pressure of the muscle compartment (Perfusion Pressure=Diastolic Blood Pressure−Compartment Pressure). The monitor can ensure the delivery of the specified functional performance needed to reliably operate the apparatus and corresponding system.

A preferred monitor, as described herein, can be provided as a single housing that incorporates a vacuum source, a pressure monitor, a blood pressure monitor, and a user interface. The vacuum source can preferably draw a vacuum at any desired level, e.g., up to 100, 200, 300, 400 or 500 mm Hg, to the interstitial fluid collection line of the apparatus. The vacuum level can be set to specific values decided upon by the physician. In addition, the module can be set to provide constant vacuum or intermittent vacuum, in order to maximize fluid removal and other parameters. The module works with pressure sensors located in the apparatus, to monitor the fluid pressure within the muscle compartment.

The monitor's user interface includes a touch screen display input to allow the user to add patient information, start and stop the procedure, and to save the data to a data storage device. The monitor will display the current compartment pressure and perfusion pressure for each catheter, along with a historical chart of the pressure from the start of the procedure.

An apparatus of this invention has particular utility for use in the field, e.g., in the course of far-forward combat medical care to reduce the mortality and morbidity associated with major battlefield wounds and injuries. For instance, such an apparatus can provide improved care for tibia fracture injuries at risk for compartment syndrome.

An apparatus of this invention can be provided and used within a corresponding system for monitoring compartment syndrome, e.g., including a pressure monitoring module and a plurality of multifunctional percutaneous catheters. In a preferred embodiment, the catheters can be used to remove interstitial tissue fluid by tissue ultrafiltration, through the hollow fiber membranes, which can contribute to a reduction in compartment pressure, and which can be used for biochemical analysis to determine whether indicators of muscle injury (biomarkers) in the analyte can be predictive of compartment syndrome development.

In use, a monitoring module can be used to sense, display and record IMP as measured by the PMFC catheter as well as provides the vacuum source that is required for the removal of fluid by both types of catheters. A monitoring module can be used to ensure the delivery of the specified functional performance requirements needed to reliably operate both catheters. In a further preferred embodiment, the catheter and monitor can be used for immediate or continuous measurement of intracompartmental pressures and/or the withdrawal of fluid for subsequent analysis.

In turn, the removal of interstitial fluid by the use of the apparatus can lower muscle compartment pressure and impact other measures of the patient's clinical status. In use, for instance, a patient can be treated by having one or more catheter portions, as described herein, inserted at the end of a surgical procedure to stabilize the tibia fracture. The catheters can be used for the first 24-hours after surgical fixation of the affected lower leg in order to: (1) measure and record muscle compartment pressure, and (2) remove accumulated interstitial fluid in the patient. Patients can receive one or more, preferably 2 or more, and more preferably 3 or more individual catheters placed in the anterior compartment. The catheter and monitor typically meet all safety and performance requirements, including biocompatibility per ISO 10993 guidelines, and sterility in accordance with established standards such as ANSI/AAMI/ISO 11135. The catheters are constructed to as to withstand normal use, including tensile strength at selected bond joints, bend testing and leak testing; and have been verified to provide pressure measurement accuracy and fluid removal capacity.

A catheter of this invention can be safely and effectively placed in a targeted muscle compartment, preferably by use of an introducer into the targeted muscle compartment, and once in position, the catheter can measure muscle compartment pressure, while also safely and effectively withdraw interstitial fluid from a muscle compartment by applying a small vacuum without causing tissue damage, and can be continuously infused to maintain patency without raising the muscle compartment pressure.

In a particularly preferred embodiment, an apparatus and corresponding system of the present invention provides an optimal combination of various features, including: safety and efficacy when positioned and used within a targeted muscle compartment; to measure muscle compartment pressure; safety and efficacy in the course of withdrawing interstitial fluid from a muscle compartment; and the ability to be continuously infused to maintain patency without raising the muscle compartment pressure.

An apparatus, including catheter portion, of this invention has demonstrated safety of use and functionality for its intended purpose. In experimental use, catheters of this invention were shown to not result in an increase in IMP and actually demonstrated a reduction in IMP as compared to the patients monitored alone. In addition, the catheter of this invention permitted the measurement of enzymes in the tissue fluid removed, which in turn has the potential to improve diagnosis of compartment syndrome. In turn, it can be seen that for patients who have had operative stabilization of a tibia fracture, use of an apparatus as described herein, providing both monitoring as well as active fluid removal, can provide a reduction in muscle compartment pressure compared to monitoring alone.

Given its various attributes, an apparatus of this invention has particular utility for use with extremity injuries of the type that represent the majority of battlefield injuries. Due to tactical problems, injured soldiers often receive life-saving measures, wound debridement, and external fixation of fractures immediately after injury, and but then undergo aeromedical evacuation to a remote location. Under these conditions, and given the severe nature of current battlefield extremity injuries, the risk of compartment syndrome commonly exists. Military medical personnel need a means to improve the diagnosis and treatment of compartment syndrome in order to minimize the need for prophylactic fasciotomy and the incidence of incompletely performed and delayed fasciotomy.

The system and apparatus of this invention can improve the care of injured warfighters at risk of compartment syndrome, since it provides clinical utility of tissue ultrafiltration in both the near- and long-term. The system improves longer term outcomes as compared to just pressure monitoring in a population of civilians that is representative of warfigher injuries, and has the potential to represent a new standard of care for military extremity injuries.

The system and apparatus of this invention can also provide benefit to extremity trauma victims, caused due to motor vehicle accidents, falls, burns, crush injuries, and periods of ischemia. For fractures alone, and can serve to reduce errors in the diagnosis of compartment syndrome, reduce the incidence of compartment syndrome, and reduce morbidity of treatment.

In a particularly preferred embodiment, a system of this invention can comprise at least four major components, namely, one or more introducers, one or more pressure monitoring and fluid collection (PMFC) catheters, one or more fluid collection (FC) catheters, and at least one compartment pressure monitor.

In use, the PMFC apparatus includes an insertable catheter portion designed to monitor muscle compartment pressure as well as facilitate excess fluid removal. It can be provided as a sterile disposable apparatus that comprises the catheter portion, one or more hollow fiber membranes adapted to be positioned within a tissue site, a pressure sensor (e.g., fiber optic type), a vacuum line and pressure sensor connectors, a catheter connection manifold, and a fluid sampling chamber with collection port.

The PMFC catheter preferably contains a pressure sensor at the distal tip that measures compartment pressure. The sensor is connected to the monitor module, e.g., by an optical fiber that extends through the entire length of the catheter. A fiber optic connector is located at the proximal end of the catheter that connects to the monitor module.

In one preferred embodiment, interstitial fluid is removed through the hollow fiber membrane located at the distal section of the catheter. Fluid passes through the walls of the micro-porous membrane, through the catheter body and into the fluid collection chamber, which in turn, is connected to a vacuum line, which connects to the monitor module. The monitor module, in turn, provides a low, intermittent or constant vacuum to the catheter to draw fluid through the hollow membrane. Fluid that is contained in the collection chamber can be aspirated using a standard syringe through a collection port, and can be then transferred to a vial and saved for analysis. A manifold is located just proximal to the fluid collection chamber. Two or more additional FC catheters can also be connected to the manifold, e.g., using standard luer-lock connections.

An FC catheter for use in a system of this invention can be designed and used to provide additional fluid collection locations within the same compartment as a PMFC catheter. The FC catheters do not provide pressure measurement and must be used with a PMFC catheter. In a preferred embodiment, an FC catheter will typically comprise a catheter body, one or more hollow fiber membranes, vacuum line connector(s), and a fluid sampling chamber(s) with corresponding collection ports.

In a particularly preferred embodiment, for instance, interstitial fluid is removed through the hollow fiber membrane located at the distal section of the catheter portion of the apparatus. Fluid passes through the walls of the micro-porous membrane, through the catheter body and into the fluid collection chamber. The fluid collection chamber can be connected to a vacuum line, which connects to the manifold of the PMFC catheter. A preferred monitor can provide for the control of vacuum, including with respect to the level (e.g., up to 100, 200, 300, 400, or 500 mg Hg) and/or the duration (e.g., intermittent, constant, and/or other desired schedule) of vacuum, in order to draw fluid through the hollow fiber membrane(s). Fluid that is contained in the collection chamber can be aspirated using a standard syringe through the collection port, where it can then be transferred to a vial and saved for analysis.

In a preferred embodiment, a PMFC catheter of this invention can be designed and used with a corresponding monitor that senses, displays and records compartment pressure as measured by one or more PMFC catheters. In a further preferred embodiment, the monitor can measure patient blood pressure using the blood pressure cuff provided, which is used for calculating the perfusion pressure of the muscle compartment (Perfusion Pressure=Diastolic Blood Pressure−Compartment Pressure). Applicant's monitor has been successfully tested to ensure the delivery of the specified functional performance needed to reliably operate the apparatuses, including catheters described herein.

Preferably, the vacuum source can draw a predetermined vacuum level to the interstitial fluid collection line of the catheter of this system. In addition, the monitoring module is preferably be set to constant vacuum which was shown to be the optimal setting. The user interface can comprise, for instance, a touch screen display input to allow the user to add a patient ID, start/stop the procedure and to save the data to a data storage device. The monitor will display the current compartment pressure and perfusion pressure for each catheter, along with a historical chart of the pressure from the start of the procedure.

### Method/Example

Surgical Procedure: The surgical procedure performed to stabilize the fracture is at the discretion of the attending surgeon. Following surgery, the leg is to be kept elevated at the level of the heart, with application of a loose compression dressing (such as an ACE bandage), and splinting to control the position of the ankle in neutral dorsiflexion.

Perioperative Medical Care—patients will typically receive, as medically indicated, appropriate hydration, pain management, and other medical care as dictated by their clinical status and institutional policies.

One or more PMFC catheters can be inserted into the injured leg in the anterior compartment at the end of the surgical procedure to stabilize the tibial fracture and then will be connected to the pressure monitor. One PMFC catheter will be inserted near the fracture site, while two other FC catheters will be inserted so that there is at least 5 cm of space between the catheters. In general, the catheters should be inserted from proximal to distal at an angle of 45 degrees. For tibial plateau fractures, it may be necessary to insert the PMFC catheter from distal to proximal; the other two FC catheters may be inserted from proximal to distal.

Catheters can be located in any suitable manner and locations, for instance:

Proximal Third Fractures: A PMFC catheter can inserted first, with its tip deep in the anterior compartment muscle, within 5 cm of the primary fracture line. The two FC catheters can then be inserted in the anterior compartment; one 5 cm distal to the PMFC catheter and one 10 cm distal to the PMFC catheter.

Middle Third Fractures: A PMFC catheter can be inserted first, with its tip deep in the anterior compartment muscle, within 5 cm of the primary fracture line. The two FC catheters can be inserted in the anterior compartment; one 5 cm distal to the PMFC catheter and one 5 cm proximal to the PMFC catheter.

Distal Third Fractures: The PMFC catheter can be inserted first, with its tip deep in the anterior compartment muscle, within 5 cm of the primary fracture line. The two FC catheters can be inserted in the anterior compartment; one 5 cm proximal to the PMFC catheter and one 10 cm proximal to the PMFC catheter.

Patient Monitoring—the patient will receive standard medical care for compartment syndrome monitoring i.e., muscle compartment pressure monitoring by catheter as well as standard clinical management of the condition. The patient's length of initial hospital stay will be determined by the attending physician based on the patient's medical condition(s). The standard-of-care for routine compartment pressure monitoring will apply to both the vacuum/non-vacuum groups in this Study with the catheters being left in place for 24 hours. Compartment syndrome is a diagnosis that depends on clinical assessment supplemented by pressure measurement. Throughout the treatment protocol, the treating physicians will monitor the patients according to standard clinical practice and will apply standard clinical judgment regarding the diagnosis of compartment syndrome. Treating physicians may perform fasciotomy if they feel it is necessary because of clinical suspicion that compartment syndrome is developing.

Blood pressure can be measured in any suitable manner, e.g., by means of a reusable BP cuff In a preferred embodiment, a module and interface for use in this invention permits the collection of blood pressure automatically by selecting the “automatic” option via the pressure monitor. In the automatic option the blood pressure can be determined at preset increments, e.g., every 2 hours (+/−30 minutes), 4 hours (+/−30 minutes), 8 hours (+/−1 hour), 16 hours (+/−1 hour), and/or 24 hours (+/−2 hours) hours during the monitoring period.

Interstitial fluid collection—the amount of fluid can be estimated and recorded, e.g., at 2 hours (+/−30 minutes), 4 hours (+/−30 minutes), 8 hours (+/−1 hour), 16 hours (+/−1 hour) and 24 hours (+/−2 hours). The fluid can be withdrawn at 4 (+/−30 minutes) hours, 16 (+/31 1 hour), and the conclusion of the 24 hour (+/−2 hours) period. Fluid can be frozen for later analysis, or immediately analyzed for potential markers such as creatine kinase, myoglobin, lactate potassium, and others.

Indications for fasciotomy—fasciotomy should be performed at the discretion of the attending surgeon based on his/her clinical experience and judgment in consideration of the presenting clinical signs, symptoms, and pressure measurements.

At the conclusion of monitoring and/or fluid collection using a system of this invention, the catheter(s) can be removed and properly destroyed.

### Post Operative Care

Follow-Up 2-week and 3-month follow-up visits will be required post completion of the 24 hour monitoring period. Functional outcomes will be measured during these follow-up as well as the occurrence of any adverse events since discharge or last follow-up. Functional outcomes will include an assessment of whether any loss of motor function resulted as part of the primary injury via the Short Musculoskeletal Function Assessment (SMFA), the Kiakkonen Ankle Scale, and a heel-raise test. The heel-raise test and the Kiakokonen ankle test will be completed at the 3 month visit only.

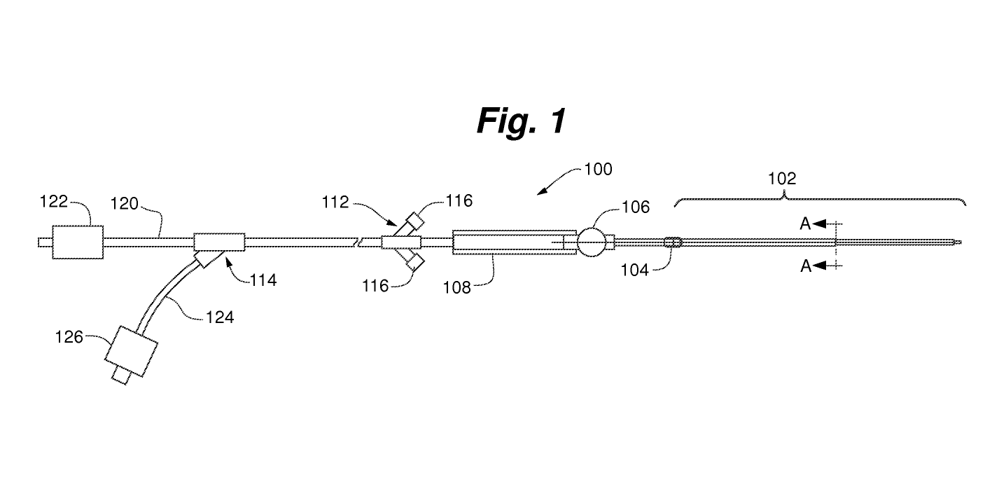

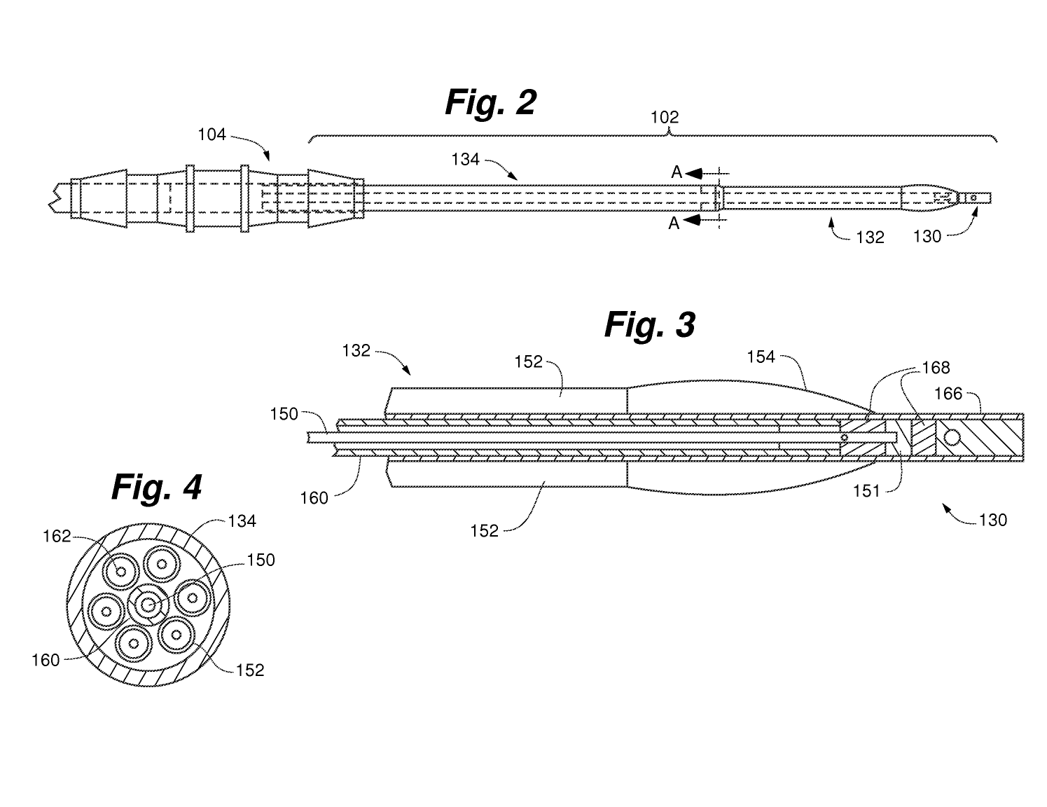

In use, patients treated with a multifiber catheter had greater fluid removal over 24 hours than those treated with single-fiber catheters (1.24+0.63 ml vs. 0.24+0.21 ml, FIG. 1). Greater fluid output was associated with lower IMP (FIG. 2). As shown in FIG. 3, among patients with single-fiber catheters, there was no difference between constant and intermittent suction (Groups A,B). In patients with multifiber catheters, constant vacuum produced greater fluid output than intermittent vacuum (1.71+0.60 ml (Group D) vs. 0.77+0.28 ml (Group C), p<0.05 by Student's T-test). In turn, the finding of lower IMP in patients with the highest volume of interstitial fluid removal supports the rationale for using TUF to lower IMP in patients with extremity injury.

Aspects of the invention will be further described with reference to the Drawings.

FIGS. 1-4 show various views and components of a preferred catheter assembly/apparatus 100 providing pressure monitoring and fluid collection (PMFC) for use in systems of the invention. As can be seen, the distal end of apparatus 100 is adapted to be inserted and positioned within the body, for use in both determining pressure within the tissue site, while also permitting the delivery and/or recovery of fluids and/or components thereof. FIG. 1 provides a side view of the catheter assembly 100 including the pressure sensor and fluid collection catheter. In particular, FIG. 1 provides a side perspective showing the overall apparatus 100, including a distal length 102 configured for insertion (shown in detail in FIG. 2), as well as proximal portions for use in providing physical and functional control as well as fluid and other communication within and between the various portions. The inserted distal end 102 is connected to the remaining portions of the assembly 100 with a barbed fitting 104 (e.g., comprising polycarbonate) at the proximal end of the inserted length 102. The fitting 104 connects the inserted length 102 to an intermediate tube (e.g., polyurethane), which in turn is connected to an injection port fitting 106. The injection port fitting 106 is coupled to a flexible tube or chamber 108, which can act as a reservoir for collecting interstitial fluids to at least some extent. The injection fitting 106 allows removal of fluids from the chamber 108 with the use of syringe, etc., in the usual manner.

In this embodiment the flexible chamber 108 is connected (via, e.g., additional polyurethane tubing) to a four-way connector 112, which in turn is connected via a braided tubing to a y-connector 114. The four-way connector 112 can be useful for coupling additional catheters (e.g., fluid collection only catheters) as is discussed elsewhere herein. When not in use, a non-vented luer cap 116 can be affixed to the end of the unused connector ports. In some cases the connector 112 includes three female luer lock connections and a single male slip connection. The y-connector 114 allows the assembly/apparatus 100 to be coupled to one or more inputs/outputs of a monitoring system. As illustrated, the y-connector couples with tubing 120 and connector 122 which provide connectivity between a pressure sensor in the distal inserted length 102 and a monitor (not shown in FIG. 1). The y-connector 114 also connects to vacuum tubing 124 and a second connector 126, which provides connectivity between a monitor's vacuum port and the fluid collection portions of the assembly 100.

FIG. 2 provides a detailed view of the distal section 102 of the assembly 100 of FIG. 1, and FIG. 3 provides a detailed view of the distal end 130 of the assembly 100. The inserted distal length 102 includes a number of separate portions, generally including the distal end or tip 130, an intermediate section 132 including one or more hollow membranes, and a proximal section 134 comprising tubing of suitable strength, flexibility, etc., for connecting the intermediate and distal portions to the rest of the catheter assembly via fitting 104. In some cases the proximal section tubing 134 may comprise a polyimide material. Referring to FIG. 3, an optical fiber 150 can be seen positioned at the center of the intermediate section 132, ultimately terminating at the catheter tip 130, where the optical fiber 150 is connected with an optical pressure sensor 151. The intermediate portion 132 includes multiple (in this case six) hollow membranes 152, which surround the optical fiber 150, extending from the tubing 134 to the tip 130.

The hollow membranes or fibers 152 typically comprise a semipermeable material, providing fluid communication between the interior of the assembly 100 (e.g., the interior of polyimide tubing 134) and the exterior environment. The hollow membranes are typically coupled to the tubing 134 by inserting the proximal ends of the hollow fibers 152 a short distance into the tubing 134 and fixing them in place with an epoxy or other adhesive, without occluding the open proximal ends of the fibers 152. At the distal tip 130, the hollow membranes 152 are coupled together with an epoxy 154, which also secures the end of the optical fiber 150 and sensor 151. In this example, the optical fiber 150 is positioned within a protective tubing 160, which extends the length of the distal inserted section 102 (e.g., extending from the barb fitting 104 to the epoxy seal 154 at the distal tip 130). In some cases the protective tubing 160 comprises a stainless steel hypotube, which surrounds the optical fiber and separates and contains the fiber 150 in case the glass fiber breaks or is otherwise damaged. FIG. 4 provides a cross-sectional view of the distal inserted section 102 taken along line A-A as shown in FIGS. 1 and 2. As seen in the cross-section, in some cases one or more hollow fibers 152 (each in this case) contains a wire 162 for strengthening and stiffening the fibers 152, while also allowing fluid flow through the fibers. Referring again to FIG. 3, in this embodiment the distal tip 130 also includes a protective sheath or sleeve 166, which extends distally from the tip, and which can incorporate slits or other openings to provide fluid communication to the pressure sensor. The pressure sensor 151 is positioned within the sleeve 166 and surrounded by a silicone gel 168, which insulates and protects the sensor 151, while still transferring pressures to the sensor 151 from interstitial fluids entering the sleeve 166 from the surrounding body.

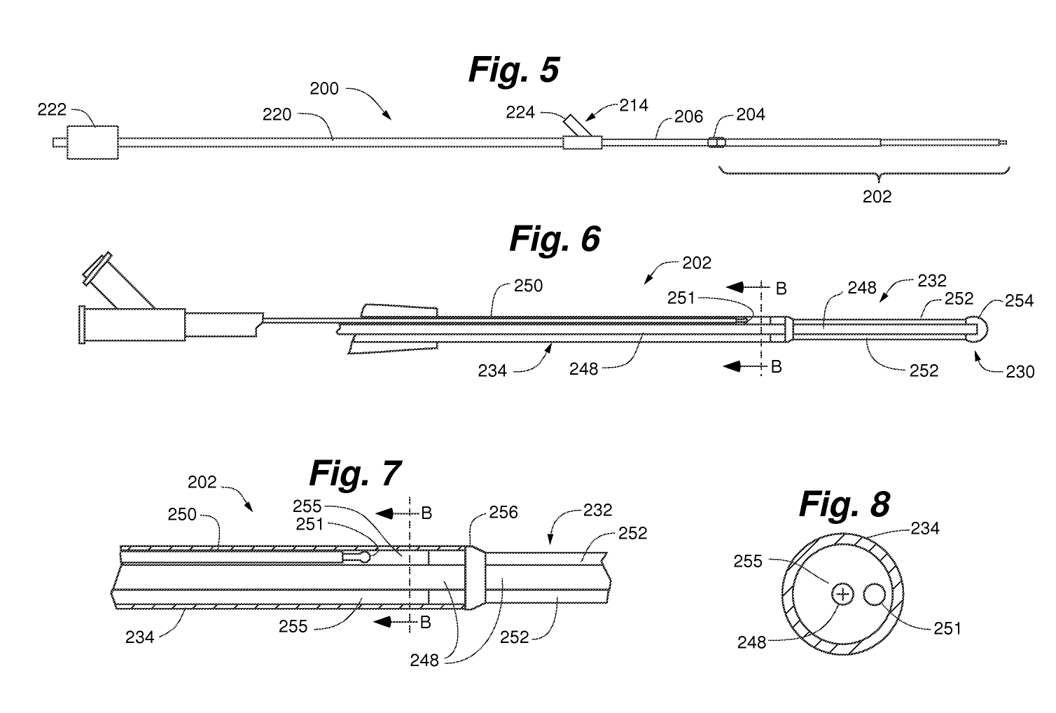

FIGS. 5-8 provide a number of views of an alternative catheter assembly 200 configured for fluid collection with multiple hollow fibers and oxygen sensing with an integrated oxygen sensor. In particular, FIG. 5 provides a side view of the catheter assembly 200, which includes a distal length 202 configured for insertion (shown in detail in FIG. 6), as well as proximal portions for use in providing physical and functional control as well as fluid and other communication within and between the various portions. The inserted distal end 202 is connected to the remaining portions of the assembly 200 with a fitting 204 at the proximal end of the inserted length 202. The fitting 204 connects the inserted length to an intermediate tube 206 (e.g., made from polyurethane), which in turn is connected to a y-connector 214. The y-connector 214 allows the assembly/apparatus 200 to be coupled to one or more inputs/outputs of a monitoring system. As illustrated, the y-connector couples with tubing 220 and connector 222 which provide connectivity between an oxygen sensor in the distal inserted length 202 and a monitor (not shown in FIGS. 5-8). The y-connector 214 also includes a vacuum branch 224, which provides connectivity between a monitor's vacuum port and the fluid collection portions of the assembly 200.

In some instances, it is preferable to monitor the physiological status of the tissue fluid after collection in the hollow fibers from the tissue site. In this manner, rather than using a single physiological sensor to obtain a localized value of the tissue parameter of interest, the tissue fluid that is collected with the hollow membrane fibers can instead represent an aggregate physiological value of the tissue site, since the fluid being collected comes from a larger volume of the tissue site as compared to a single point source contact. In this way, one or more individual sensors can provide the physiological status of the corresponding tissue site(s) as compared to an isolated value from the tissue contact.

FIGS. 6 and 7 provide detailed views of the distal section 202 of the catheter assembly 200, while FIG. 8 provides a cross sectional view taken along line B-B of the assembly 200 shown in FIGS. 6 and 7. The inserted distal length 202 includes a number of separate portions, generally including the distal end or tip 230, an intermediate section 232 including one or more hollow membranes, and a proximal section 234 comprising tubing of suitable strength, flexibility, etc., for connecting the intermediate and distal portions to the rest of the catheter assembly via fitting 204. Referring to FIGS. 6 and 7, a stylet or wire 248 can be seen positioned at the center of the intermediate section 202, ultimately terminating at the catheter tip 230 within an epoxy member 254. The portion 232 includes multiple hollow membranes 252, which surround the stylet 248, extending from the tubing 234 to the tip 230. As discussed above, the hollow membranes or fibers 252 may comprise a semipermeable material providing fluid communication between the interior and exterior of the assembly 200. As shown in FIG. 7, the hollow membranes 252 are coupled to the tubing 234 with the proximal ends of the hollow fibers 252 inserted into the tubing 234 and fixed in place with an epoxy 256. At the distal tip 230, the hollow membranes 252 are coupled together about the stylet 248 within the epoxy member 254. In this example, a sensor wire/conductor 250, terminating with an oxygen sensor 251, is positioned within the fluid path 255, which is defined by the tubing 234 and the stylet 248 positioned within the tubing. FIG. 8 provides a cross-sectional view of the tubing 234 just distal to the oxygen sensor 251. Although not visible in FIGS. 5-8, the sensor wire 250 extends proximally through the catheter assembly 200 and terminates at the sensor connector 222.

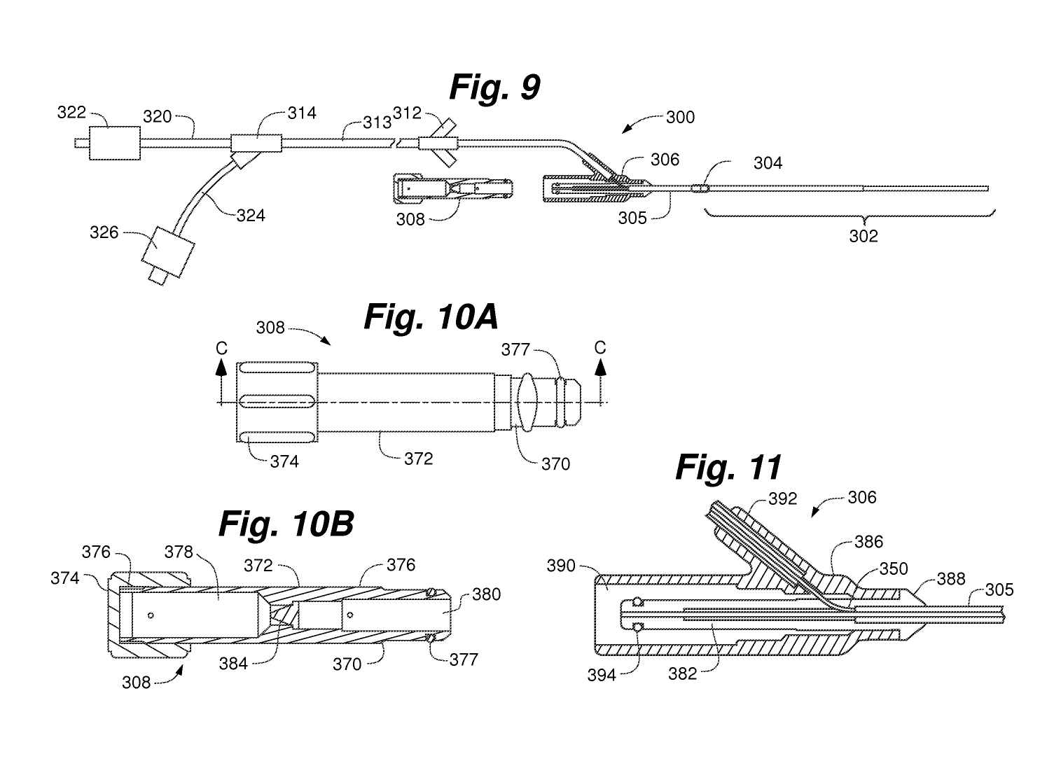

FIGS. 9-11 illustrate multiple views of another catheter assembly 300 having a removable cartridge according to an embodiment of the invention. The catheter assembly 300 includes a pressure sensor (not shown) and fluid collection catheter, and is similar in many respects to the catheter assembly/apparatus 100 illustrated in FIGS. 1-4. In particular, the catheter apparatus 300 includes a distal length 302 configured for insertion, as well as proximal portions for use in providing physical and functional control as well as fluid and other communication within and between the various portions. In this example, the inserted distal portion 302 the same as the distal portion 102 illustrated in FIGS. 2-4. The inserted distal end 302 is connected to the remaining portions of the assembly 300 with a fitting 304 (e.g., comprising polycarbonate) at the proximal end of the inserted length 302. The fitting 304 connects the inserted length 302 to an intermediate tube 305, which in turn is connected to a removable cartridge manifold 306. As will be discussed in more detail, the removable cartridge manifold is configured to receive a removable cartridge 308, which can act as a reservoir for collecting interstitial fluids to at least some extent. Instead of sampling fluids within the reservoir with a syringe, the cartridge 308 can be removed from the manifold 306 for assaying.

As with the embodiment in FIGS. 1-4, the catheter assembly 300 includes a four-way connector 312, although this is not required. The connector 312 is coupled via a braided tubing 313 to a y-connector 314. As illustrated, the y-connector couples with tubing 320 and connector 322 which provide connectivity between the pressure sensor in the distal inserted length 302 and a monitor (not shown in FIG. 9). The y-connector 314 also connects to vacuum tubing 324 and a second connector 326, which provides connectivity between a monitor's vacuum port and the fluid collection portions of the assembly 300.

FIG. 10A provides a detailed view of the removable cartridge 308 and FIG. 10B provides a cross sectional view taken along line C-C shown in FIG. 10A. The removable cartridge 308 is generally constructed from an interior cartridge body 370 that is surrounded by a tube wall 372. An end cap 374 with ridges or other surface features affixed to the proximal end allows for easy removable of the cartridge from the manifold 306. As shown in FIG. 10B, an adhesive 376 can be used to attach the components of the cartridge together. The cartridge 308 can be formed from any suitable material. One example is a polymer, such as PET. The distal end of the cartridge 308 includes an o-ring 377 (e.g., made from silicone), which provides a tight seal within the manifold 306. The interior of the cartridge includes a reservoir 378 into which interstitial fluid can flow, and a manifold receiving cavity 380, which receives a connecting portion 382 of the manifold. A valve 384 (e.g., a duckbill check valve) positioned between the reservoir 378 and cavity 380 prevents fluid from flowing back into the manifold 306.

FIG. 11 provides a detailed view of the cartridge manifold section 306. The manifold 306 generally includes an outer form 386 and a centering pin 388 that is received and attached within the outer form 386. The outer form 386 defines a receiving cavity 390 about the centering pin 388 for receiving the removable cartridge 308. The centering pin 388 is configured to receive the proximal end of the intermediate tubing 305, direct the sensor optical fiber 350 towards an outlet branch 392, and provide fluid communication between the intermediate tubing 305, the receiving cavity 390, and the outlet branch 392. The centering pin 388 is also configured to be inserted within the receiving cavity 380 on the removable cartridge, with an additional o-ring 394 providing a seal between the centering pin and the receiving cavity of the cartridge.

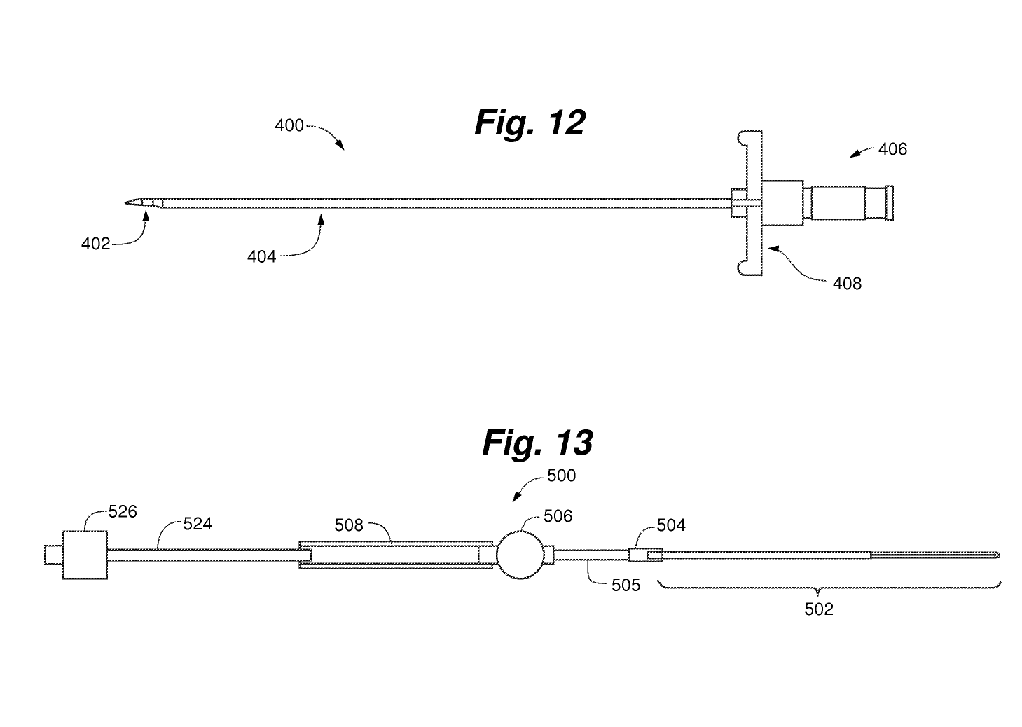

FIG. 12 provides a side view of an introducer assembly 400 for use in various embodiments of the invention. The introducer assembly 400 includes a stainless steel trocar 402 and a tear-away plastic sheath 404. The assembly 400 includes a trocar hub 406 for insertion of one or more catheters, and a sheath hub 408 connected to the tear-away sheath 404. The sterile disposable introducer 400 provides access to a targeted muscle compartment to facilitate the placement of one of the catheter assemblies described herein or other embodied aspects of the invention. IN one example, the sharp-tipped trocar 402 and sheath 404 are inserted through the skin and into the targeted muscle compartment. Once properly positioned, the trocar 402 is removed leaving the hollow tear-away sheath 404 in place. The catheter can then be placed through the hollow sheath 404 and into the muscle compartment. Once the catheter is placed, the sheath 404 is designed to easily tear away for removal. The introducer's trocar and tear-away sheath design and materials can be provided in various ways that can become apparent to those skilled in the art, e.g., the tear-away sheath can be constructed of thin walled polyethylene tubing, while the trocar can be composed of stainless steel needle with a three-facet sharp tip point.

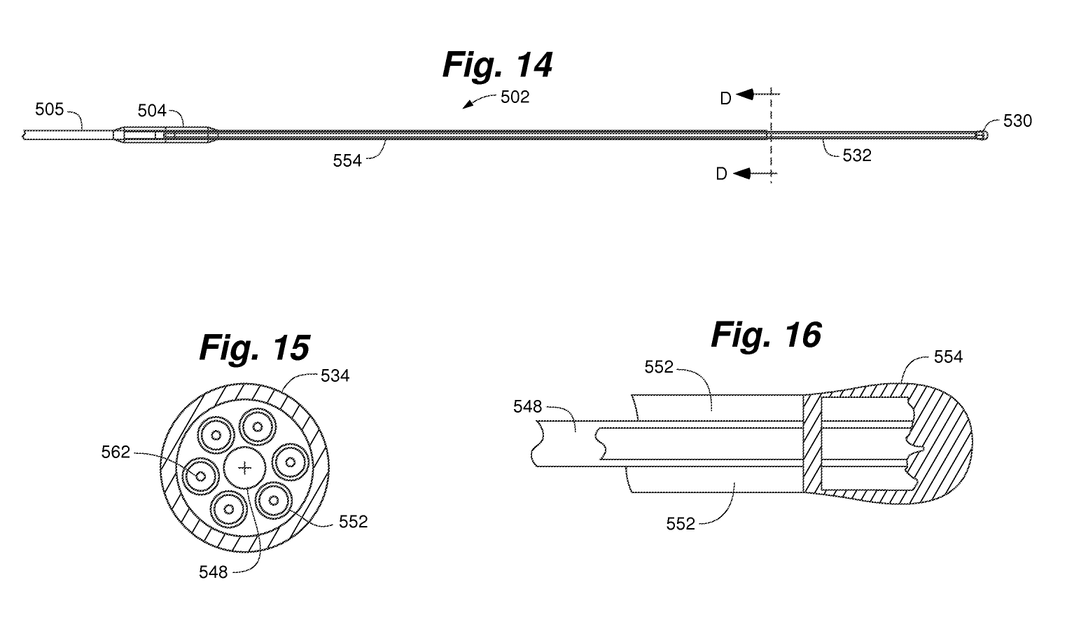

FIGS. 13-16 provides multiple views of one example of a fluid collection (FC) catheter 500 according to an embodiment of the invention. FIG. 13 provides a side view of the FC catheter 500. The FC catheter 500 can be used in different embodiments of the invention to provide for delivering and/or withdrawing fluid and/or fluid components to or from the tissue site, and may or may not be used in conjunction with one or more other FC catheters and/or with a PMFC (pressure monitoring and fluid collection) catheter. In particular, FIG. 13 provides a side perspective showing the catheter 500, including a distal length 502 configured for insertion (shown in detail in FIGS. 14 and 16), as well as proximal portions for use in providing physical and functional control as well as fluid and other communication within and between the various portions. The inserted distal end 502 is connected to the remaining portions of the catheter 500 with a barbed fitting 504 (e.g., comprising polycarbonate) at the proximal end of the inserted length 502. The fitting 504 connects the inserted length 502 to an intermediate tube 505 (e.g., comprising polyurethane), which in turn is connected to an injection port fitting 506. The injection port fitting 506 is coupled to a flexible tube or chamber 508, which acts as a reservoir for collecting interstitial fluids. The injection fitting 506 allows removal of fluids from the chamber 508 with the use of syringe, etc., in the usual manner. In this embodiment the flexible chamber 508 is connected to vacuum tubing 524 and a connector 526 (e.g., a Luer connector). In some circumstances the connector 526 allows the catheter 500 to be coupled to a vacuum port of a monitor. In some cases the connector 526 also/alternatively allows the catheter 500 to be coupled with one or more other catheter assemblies through a common vacuum connection. For example, the catheter 500 could be coupled to one of the catheter assemblies 100, 300 through one of the branches of the four-way connectors 112, 312.

FIG. 14 provides a detailed view of the distal section 502 of the catheter 500, and FIG. 16 provides a detailed view of the distal end 530 of the catheter 500. The inserted distal length 502 generally includes the distal end or tip 530, an intermediate section 532 including multiple hollow membranes 552, and a proximal section 534 comprising tubing of suitable strength, flexibility, etc., for connecting the intermediate and distal portions to the rest of the catheter assembly via fitting 504. In some cases the proximal section tubing 534 may comprise a polyimide material. Referring to FIGS. 16 and 15 (providing a cross-section along line D-D), a stylet 548 is positioned at the center of the distal inserted length 502, ultimately terminating at the catheter tip 530 within an epoxy member 554. The portion 532 includes multiple hollow membranes 552, which surround the stylet 548, extending from the tubing 534 to the tip 530. As discussed above, the hollow membranes or fibers 552 may comprise a semipermeable material providing fluid communication between the interior and exterior of the catheter 500. As shown in FIG. 16, the hollow membranes 552 are coupled together about the stylet 548 within the epoxy member 554 at the distal tip 530. As seen in the cross-section of FIG. 15, in some cases one or more hollow fibers 552 contains a wire 562 for strengthening and stiffening the fibers 552, while also allowing fluid flow through the fibers.

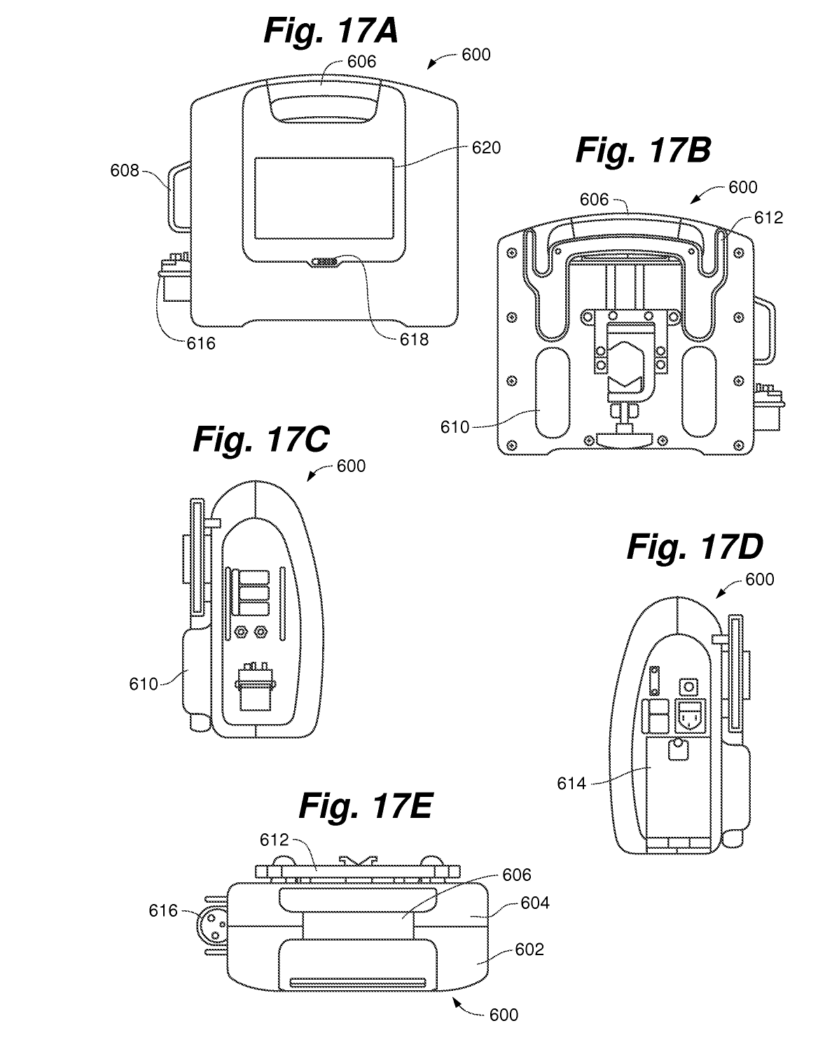

FIGS. 17A-17E provide views of a catheter assembly monitor 600 according to an embodiment of the invention. In this embodiment the monitor 600 includes a two-part main enclosure comprising a front part 602 and a rear part 604. Other physical attributes/components of the enclosure include, but are not limited to, a handle 606, a protective cable connection bars 608, rear ridges 610 to support the enclosure against a bed, wall, etc., a passive cable management feature 612, a battery access panel 614, and a reservoir holder 616. The monitor 600 also includes a battery level indicator 618, and a touch-screen display 620 that displays relevant information and allows interaction with a user interface program. In a preferred embodiment, the monitor 600 incorporates a vacuum source, a pressure monitor, a blood pressure monitor, and a user interface. An oxygen sensor and/or any other desired sensing capabilities can also be included as needed.

Catheter assemblies/apparatuses of the invention are designed to be used with the monitor 600, which can sense, display and record compartment pressure as measured by one or more such catheter assemblies. In addition, the monitor 600 can be used to measure patient blood pressure using a cuff provided, which is used for calculating the perfusion pressure of the muscle compartment (Perfusion Pressure=Diastolic Blood Pressure−Compartment Pressure). The monitor can ensure the delivery of the specified functional performance needed to reliably operate the apparatus and corresponding system. In a preferred embodiment, the monitor 600 includes a single housing that incorporates a vacuum source, a pressure monitor, a blood pressure monitor, and a user interface. In some cases the vacuum source can draw a variable or other predetermined vacuum on the interstitial fluid collection line of one or more connected catheters. The vacuum level can be set to specific values decided upon by the physician. In addition, the monitor 600 can be set to provide constant vacuum or intermittent vacuum, in order to maximize fluid removal and other parameters. The module works with pressure sensors located in the apparatus, to monitor the fluid pressure within the muscle compartment. The module's user interface includes a touch screen display input 620 to allow the user to add patient information, start and stop the procedure, and to save the data to a data storage device. The monitor 600 will display the current compartment pressure and perfusion pressure for each catheter, along with a historical chart of the pressure from the start of the procedure. Applicant's own previous patents and applications, including for instance U.S. Ser. No. 10/508,610 (Publication No. 2005-0165342), describe, inter alia, various manners in which suitable monitors such as monitor 600 can be used in an integrated fashion with catheters having semipermeable membranes.

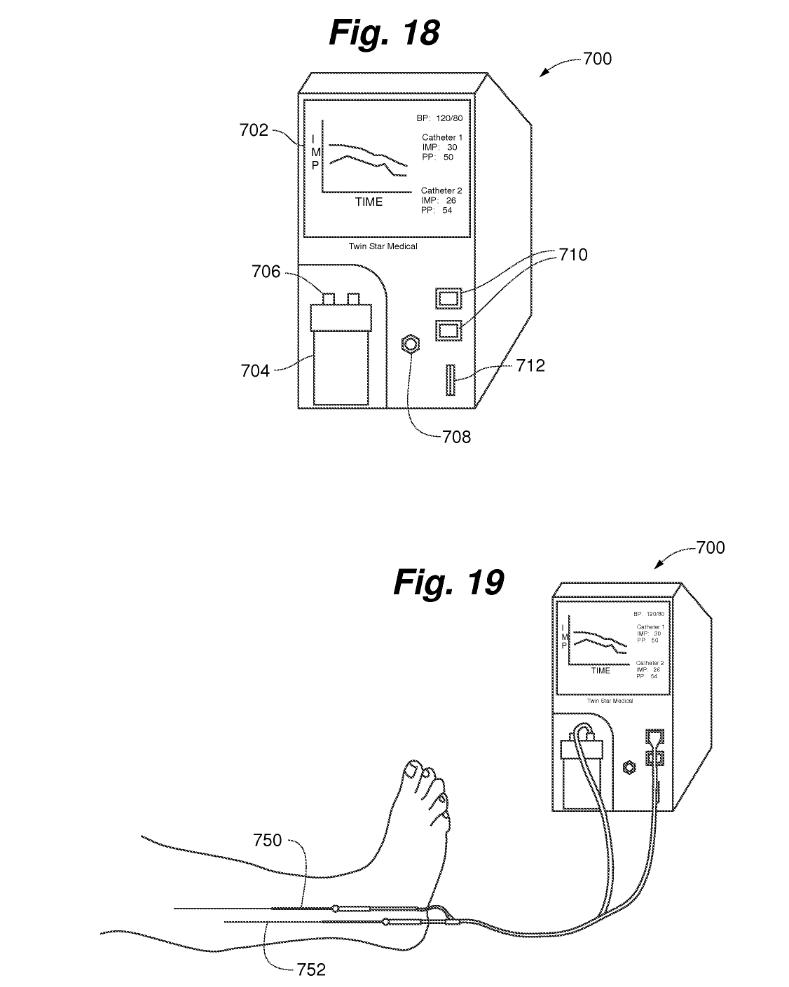

FIG. 18 provides a view of another catheter assembly monitor 700 according to an embodiment of the invention. The monitor 700 can include any of the functionalities described with respect to the monitor 600 shown in FIGS. 17A-17E. In particular, the monitor 700 includes an LCD display/touchscreen 702, a fluid overflow reservoir 704 with catheter vacuum connection ports 706, a blood pressure cuff connection 708, catheter pressure connection ports 710, and a USB data port 712. FIG. 19 provides a view of the catheter assembly monitor 700 in use in combination with multiple catheters 750, 752 according to an embodiment of the invention.

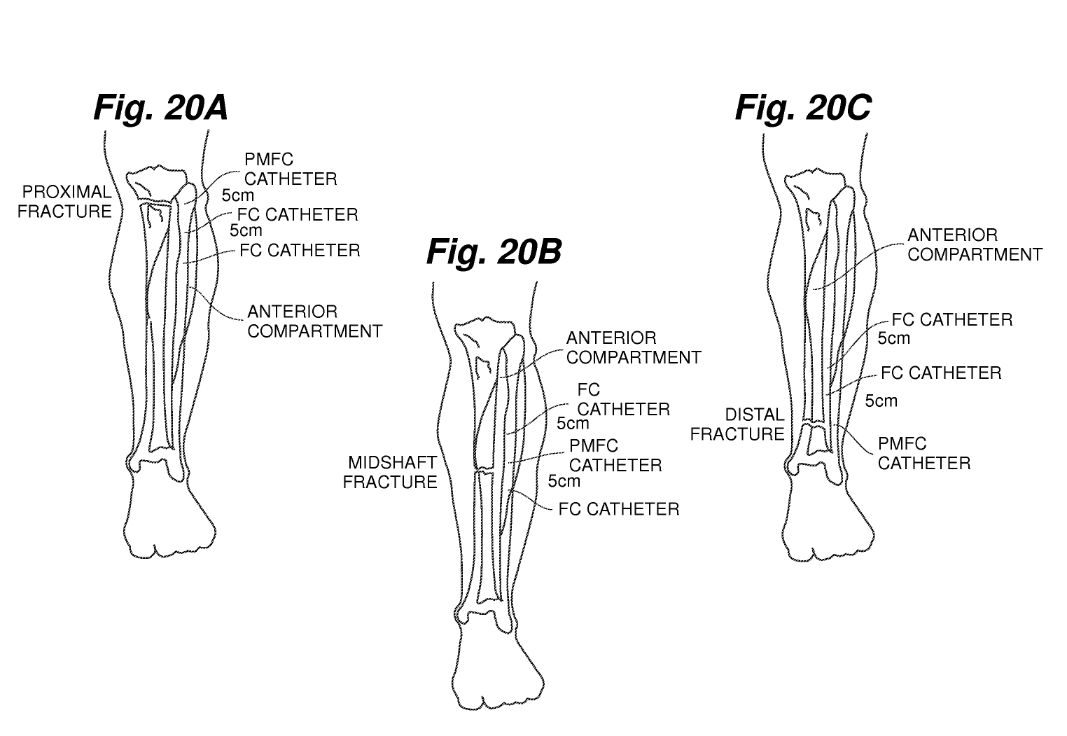

FIGS. 20A-20C provide front views of multiple catheter placements for various tibia fracture settings. In particular, FIG. 20A illustrates a set-up configuration for intra-compartment pressuring monitoring of proximal third fractures. In this case, a PMFC catheter can be inserted first, with its tip deep in the anterior compartment muscle, within 5 cm of the primary fracture line. Two FC catheters can be inserted in the anterior compartment, one 5 cm distal to the PMFC catheter and one 10 cm distal to the PMFC catheter. FIG. 20B illustrates a set-up configuration for intra-compartment pressuring monitoring of middle third fractures. In this configuration, the PMFC catheter is inserted with its tip deep in the anterior compartment muscle, within 5 cm of the primary fracture line. Two FC catheters are inserted in the anterior compartment, one 5 cm distal to the PMFC catheter and one 5 cm proximal to the PMFC catheter. FIG. 20C illustrates a set-up configuration for intra-compartment pressuring monitoring of distal third fractures. In this configuration, the PMFC catheter is inserted with its tip deep in the anterior compartment muscle, within 5 cm of the primary fracture line. Two FC catheters are inserted in the anterior compartment, one 5 cm proximal to the PMFC catheter and one 10 cm proximal to the PMFC catheter.

What is claimed is:

1. A system comprising:
a) at least one apparatus comprising a catheter portion that comprises
i) a functional device tip adapted to be positioned and used within a tissue site that comprises both fluid and non-fluid tissue, and
ii) one or more hollow fiber membranes adapted to be positioned within the tissue site in order to collect fluid therefrom,
the apparatus being adapted to be safely inserted and positioned within the tissue site, in a manner that permits it to function there while substantially minimizing direct impingement of the non-fluid tissue on the functional device tip surface,
the catheter comprising a distal insertable portion that comprises the functional device tip contained within a protective catheter, together with a plurality of hollow fiber membranes, and
b) a monitor adapted to monitor and control the function and operation of the apparatus(es).

2. A system according to claim 1 wherein the catheter comprises an insertable portion that comprises a pressure sensor, contained within a protective catheter, which is in turn surrounded by an axial array of hollow fiber members.

3. A system according to claim 1, wherein the system further comprises one or more apparatuses comprising catheter portions adapted to inserted into the tissue site in order to remove additional fluid from the tissue site.

4. A system according to claim 3 wherein the fluid collection catheter(s) are adapted to be used in combination with the pressure monitor/fluid collection catheter, in that they are adapted to be positioned in corresponding tissue sites and operated together by use of the monitor in a manner that optimizes treatment.

5. A system according to any previous claim, further comprising an apparatus comprising an insertable catheter portion comprising an oxygen sensor.

6. A system according to any previous claim wherein the system comprises one or more apparatuses for both monitoring pressure and for fluid collection, and one or more apparatuses for fluid collection, the apparatuses being adapted to be inserted and used in order to collect fluid from a common tissue site, through the respective hollow fibers and by means of a single vacuum source.

7. A system according to any previous claim, wherein one or more of the apparatus(es) comprise a removable cartridge for fluid collection using the apparatus.

8. A system according to any previous claim, wherein the monitor provides one or more features selected from the group consisting of: a) a self zeroing pressure transducer, b) one or more pressure alarms, c) wireless communication capability, d) data transmission capability, e) renewable and/or replaceable power sources, f) all in one utility and mounting, and g) accessory USB inputs.

9. A system comprising:
a) at least one apparatus comprising a catheter portion that comprises
i) a functional device tip adapted to be positioned and used within a tissue site that comprises both fluid and non-fluid tissue, and
ii) one or more hollow fiber membranes adapted to be positioned within the tissue site in order to collect fluid therefrom,
the apparatus being adapted to be safely inserted and positioned within the tissue site, in a manner that permits it to function there while substantially minimizing direct impingement of the non-fluid tissue on the functional device tip surface,
the catheter comprising a distal insertable portion that comprises the functional device tip contained within a protective catheter, together with a plurality of hollow fiber membranes,
b) a monitor adapted to monitor and control the function and operation of the apparatus(es), and
c) at least one apparatus comprising a catheter portion adapted to remove additional fluid from the tissue site,
the apparatuses being adapted to be inserted and used in order to collect fluid from a common tissue site, through the respective hollow fibers and by means of a single vacuum source.

10. A system according to claim 9 wherein the apparatuses comprise a common, removable cartridge for fluid collection.

11. A system according to claim 9 wherein the monitor can be connected to the apparatus(es) connected at any time, without the need to first zero the monitor with respect to atmospheric pressure.

12. An apparatus comprising a catheter portion that comprises
a) a functional device tip adapted to be positioned and used within a tissue site that comprises both fluid and non-fluid tissue, and
b) one or more hollow fiber membranes adapted to be positioned within the tissue site in order to collect fluid therefrom,
the apparatus being adapted to be safely inserted and positioned within the tissue site, in a manner that permits it to function there while substantially minimizing direct impingement of the non-fluid tissue on the functional device tip surface,
the catheter comprising a distal insertable portion that comprises the functional device tip contained within a protective catheter, together with a plurality of hollow fiber membranes.

13. A method of treating or preventing compartment syndrome, the method comprising the steps of providing a system according to claim 1, and positioning the insertable distal end of the catheter into a tissue site exhibiting compartment syndrome in order to both monitor pressure and withdraw fluids.
